(19) United States
(12) Patent Application Publication
(10) Pub. No.: US 2019/0099445 A1
(43) Pub. Date: Apr. 4, 2019

(54) A PHARMACEUTICAL OR VETERINARY COMPOSITION

(71) Applicant: Epsilion Limited, Tralee, County Kerry (IE)

(72) Inventors: Gearoid SAYERS, Tralee, County Kerry (IE); Lea KRUMP, Tralee, County Kerry (IE)

(73) Assignee: Epsilion Limited, Tralee, County Kerry (IE)

(21) Appl. No.: 16/086,910

(22) PCT Filed: Mar. 21, 2017
(86) PCT No.: PCT/EP2017/056747

§ 371 Date: Sep. 20, 2018

(30) Foreign Application Priority Data
Mar. 21, 2016 (GB) 1604765.6
Mar. 22, 2016 (GB) 1604888.6

(51) Int. Cl. A61K 33/10; A61K 47/22; A23K 20/22; A23K 20/10; A61K 9/00; A23K 50/10; A61P 1/12

(52) U.S. Cl. CPC A61K 33/10; A61K 47/22; A23K 20/22; A61K 31/7004; A61K 9/0053; A23K 50/10; A61P 1/12; A23K 20/10

(57) ABSTRACT

The invention is directed to a pharmaceutical or veterinary composition comprising, when dissolved in a diluent, a strong ion difference in the range of 165-370 mmol/l. The invention is also directed to a pharmaceutical or veterinary composition comprising, when dissolved in a diluent, an alkalizing agent, optionally bicarbonate, in the range of 130 to 370 mmol/l. The invention is also directed to a method of treating diarrhoea in a subject, optionally a domesticated animal, the method comprising administering the aforementioned compositions. The invention is also directed to the aforementioned compositions for use in a method of treating diarrhoea in a subject, optionally a domesticated animal.

This invention relates to a pharmaceutical or veterinary composition for treating diarrhoea.

Neonatal calf diarrhoea is the most common cause of mortality in calves (Azizzadeh et al., 2012; Torsein et al., 2011). Electrolyte disturbance, dehydration and metabolic acidosis, accompanied by a strong ion difference (SID), are the most significant consequences of diarrhoea in calves (Smith and Berchtold, 2014). Veterinary assessment of calves with diarrhoea is generally based on clinical examination alone, however blood gas analysis remains the most detailed approach to assess the degree of electrolyte disturbance and acidosis in diarrhoeic calves. Russell and Roussel (2007) have previously highlighted blood gas analysis as a useful tool in practice, especially combined with history and physical examination.

In many cases, initial diagnosis and treatment of neonatal calf diarrhoea is predominantly carried out by primary producers (farmer/manager), who utilise an oral rehydration and buffering solution (ORBS) as a first inexpensive attempt to address calf diarrhoea. An ORBS is recommended for a diarrhoeic calf when dehydration is less than 8% and there is evidence of a suckle reflex (Lorenz et al., 2011). The purpose of this solution (ORBS) is to promote plasma expansion, correct electrolyte imbalances, provide glucose as a co-transport partner of sodium to facilitate water resorption, and an alkalising agent to address the strong ion/metabolic acidosis (Smith, 2009). However, uncertainty remains regarding the optimal electrolyte concentrations, type of buffer, energy source and osmolality of the ideal ORBS solution (Constable et al., 2009; Naylor, 1989; Sen et al., 2009). Accordingly, a large number of ORBS products are commercially available, differentiated by composition and administration protocols (Smith and Berchtold, 2014). This makes it difficult for producers, and veterinarians, to identify a product that best suits the needs of diarrhoeic animals, including calves.

European directive, 2008/38 EC (amendment M4, 22 Oct. 2014), sets requirements and recommendations for an ORBS to be suitable for treatment of electrolyte imbalance and acidosis in calves. It emphasises a minimum SID value of 60 mM for such therapies. Based on their recommendations, the SID can range from 65 to 138 mmol/l (see table below):

| Ingredients | recommended range (g/l) | molality (mmol/l) |
|---|---|---|
| Na | 1.7-3.5 | 74-152 |
| K | 0.4-2.0 | 5-26 |
| Cl | 1-2.8 | 14-40 |
| SID | | 65-138 |

Based on the interpretation of Stewart (1981), SID is regarded as the major factor in determining the alkalising ability of an ORBS and as a valid approach when formulating an ORBS for calves with diarrhoea and metabolic derangement (Stampfli et al., 2012).

The question as to which is more important, an ORBS with high SID, or an ORBS with an alkalinising agent has yet to be definitively answered (Smith and Berchtold, 2014). There is also no consensus on a suitable alkalinising agent. The use of bicarbonate precursors such as acetate or propionate are favoured over bicarbonate for their energy value once metabolised, their water absorption capabilities and that they do not alkalinise the abomasum. Bicarbonate was believed to inhibit abomasal milk clotting, however this has not been supported by more recent studies (Bachmann et al., 2009; Constable et al., 2009).

Unlike medicines, which undergo rigorous testing prior to European Commission approval, ORBSs within Europe are not assessed for clinical efficacy before market placement. While a limited number of studies have assessed various aspects of ORBS treatment in natural neonatal calf diarrhoea (Constable et al., 2009; Grunberg et al., 2013; Kirchner et al., 2014; Naylor, 1989; Stampfli et al., 2012; Stämpfli et al., 1996), there is a lack of observational field studies in recent years examining the efficacy and suitability of ORBSs for use in calf diarrhoea (Meganck et al., 2014), in particular data relating to an ORBS conforming to recently amended European Union (EU) legislation.

In an attempt to increase scientific knowledge in this area, the aim of this observational study was to investigate outbreaks of calf diarrhoea on several dairy farms using rapid ‘pen-side’ blood gas analysis and subsequently to evaluate treatment of diarrhoeic calves using an ORBS that is compliant with current EU legislation.

Neonatal calf diarrhoea is an infectious disease of bacterial, virial or protozoal origin that is invariably followed by dehydration and metabolic acidosis. The treatment for calf diarrhoea (regardless of the cause) is based on fluid therapy, most commonly using oral rehydration solutions (ORS). These solutions usually contain the basic ingredients of glucose, sodium (Na+), potassium (K+) and chloride (Cl−). Often a buffer, such as bicarbonate or its precursor, is added to address the acidosis.

The scientific literature has recently focused on a strong ion difference (SID) approach to determine the suitability for purpose of a rehydration solution.

SID is used as a proxy for buffering capacity (Stewart, 1981). The term “SID”, as used herein, is calculated using the concentration of three ions in the solution with the following formula: [Na+]+[K+]−[Cl−]. The current scientific recommendations for SID are between 60 mmol/l (Smith and Berchtold, 2014) and 110 mmol/l (Stämpfli et al., 2012) although an optimal SID for a solution has not yet been determined.

Our research, both in vitro and in vivo, investigated alternative scientific approaches to using an oral rehydration solution. Our aim was to re-evaluate the conventional scientific knowledge on the best and most appropriate solution for the treatment of calves with diarrhoea and acidosis.

Our invention goes directly against the conventional scientific wisdom on the most suitable SID for a rehydration solution. Most of the ingredients in our product are similar to other products but their concentration and combination differs significantly. Our formula is different in the following ways:

The SID of our formula is innovative in its concentration. We took the ‘non-obvious’ approach by investigating concentrations that went well beyond the scientific recommendations—from 150 and up to 370 mmol/l. The concentration we now claim goes against the conventional wisdom and, to the best of our knowledge, our closest competitor has an SID concentration of 140 mmol/l—taken from Smith and Berchtold, 2014. We currently have the highest SID ever to be used in a product for calf diarrhoea which accounts for the efficacy.

The SID of the formula is our main focus, since it is the key to improved efficacy of our product. An investigation of the key electrolyte manufacturers (Appendix 1 below) indicates SID values all well below 100, matching the current recommendations.

To the best of our knowledge, the highest SID value of a solution ever used for research purposes was 189 mmol/l (Bachmann et al., 2009). However, to achieve this value, they had used double the concentration of the oral rehydration solution than was recommended by the manufacturer and the solution was prepared with milk replacer, which has its own SID of 42 mmol/l and osmolality of 287 mOsm/kg. Therefore, the solution prepared in water (even by doubling the concentration) would have an SID of 147 mmol/l and an osmolality of 633 mOsm/kg.

The research article by Bachmann et al. (2009) reflects ad hoc research investigating the potential negative impact of SID (of a solution) on abomasal emptying. The authors attempted to purposely stray outside the scientifically accepted norms of SID ranges and investigate extreme values to draw reasonable conclusions on this effect. They did not investigate diarrhoea but rather focused on healthy calves for a different purpose, whereas our invention was assessed in sick calves with the sole purpose of treating diarrhoea in those calves.

It is evident that “only plasma volumes of groups fed MR [milk replacer] and MR-ORS [milk replacer-oral rehydration solution] mixtures were still increased, whereas plasma volumes of groups fed water-ORS mixtures were back to baseline. The MR-ORS mixtures were most effective in increasing plasma volume at the 2 determined time points, reaching statistical significance compared with MR or water-ORS mixtures 240 min after feeding (P<0.05).” (Bachmann et al., 2009).

Therefore, Bachmann et al. (2009) have concluded that “administration of these MR-ORS mixtures causes a more pronounced expansion of plasma volume, which is beneficial for the correction of dehydration in diarrheic calves.”

In contrast to that, we have found an optional formula that has an SID of 237 mmol/l by mixing it with water alone, which has been proven to be beneficial for the correction of dehydration in calves with diarrhoea.

Indeed, subsequent analysis on this topic by the same group (Bachmann et al., 2012) focuses the research at an SID range of 35-84 mmol/L, further advancing the ad hoc nature of the 2009 investigation with an SID of 189 mmol/L which was employed to test the extremes on abomasal emptying alone. Additionally, Bachmann et al did not choose to advance studies on the high SID values but on another electrolyte (Lytafit) because “it was shown that the preparation of Lytafit in MR did not impair abomasal milk clotting”.

From this study, Bachmann et al. (2012) generates two conclusions on SID—(a) “an ORS with a [SIDS] of 84 mmol/L increased plasma [SIDS] in healthy calves, indicating that, for effective correction of metabolic acidosis in diarrheic calves, ORS should contain high [SIDS] values”, and (b) “an ORS with [SIDS] values >80 mmol/L, which expand [SID] in healthy calves, should be used in oral rehydration treatment of diarrheic calves”.

It is proposed that Bachmann's SID focus was in the range of 84 mmol/L and the use of the term ‘high’ (“ORS should contain high [SIDS] values”) was presented/stated in the context of the prevailing theory that the most favourable SID was 60-80 mmol/L (Smith and Berchtold, 2014).

The use of milk or water as an ORS diluent has been the subject of much research. From their own studies, Bachmann et al. (2009 & 2012) conclude that milk (or milk replacer) is the preferred option. It is stated by these authors that “only plasma volumes of groups fed MR [milk replacer] and MR-ORS [milk replacer-oral rehydration solution] mixtures were still increased, whereas plasma volumes of groups fed water-ORS mixtures were back to baseline. The MR-ORS mixtures were most effective in increasing plasma volume at the 2 determined time points, reaching statistical significance compared with MR or water-ORS mixtures 240 min after feeding (P<0.05).” (Bachmann et al., 2009).

In addition, the MR-ORS solution with an SID of 189 mmol/l contained acetate as the alkalising agent, which Smith and Berchtold, 2014 has demonstrated has several advantages over bicarbonate, whereas our invention optionally contains bicarbonate and has, surprisingly, been proven to be beneficial for the correction of acidosis in calf diarrhoea.

Alternatively, or additionally, the osmolality of our formula is 750 to 1300, optionally, 750 to 1000 or to 1000, further optionally, about 939 mOsm/l—this is significantly higher than that of an average ORS. According to our research, the product with the highest osmolality is Entrolyte HE (Zoetis, USA) with an osmolality of 739 mOsm/l (taken from Smith and Berchtold, 2014). However, current scientific recommendations advise against ORS with an osmolality higher than 750 mOsm/l (Nouri and Constable, 2006).

Alternatively, or additionally, the present composition comprises a significantly increased content of an alkalising agent in the range, when dissolved in a diluent, in the range of 150-370 or 130-300 or 130-370 mmol/l, for example, we used Sodium Bicarbonate as an alkalising agent at a concentration of about 237 mmol/l. The current scientific recommendation for an ORS is to contain 50-80 mmol/l of an alkalising agent (Smith, 2009) and most products on the market contain 80 mmol/l or less of an alkalising agent (Appendix 1).

The interplay between alkalising agent and SID in acid-base assessment has been the subject of significant research—as reviewed by Constable et al., 2014. The traditional approach for assessing acid-base balance in animals uses the Henderson-Hasselbalch equations and focuses on how plasma pH is determined by the interaction between carbon dioxide tension (PCO₂), the bicarbonate concentration (cHCO₃₋), the negative logarithm of the apparent dissociation constant (pK10) for carbonic acid (H₂CO₃), and the solubility coefficient for CO₂ in plasma (S). This equation, however, is limited to healthy animals with plasma protein concentrations within the reference range, and cannot be applied to neonatal calf diarrhoea. New models based on strong ions, developed by Stewart (1981) and refined by Constable (2000), established a mechanistic acid-base theory that can provide an enhanced understanding of acid-base disturbances in pathological cases. This theory indicates that it is the sodium in sodium bicarbonate and, therefore, the SID that is important in correcting the acid-base disturbance in a diarrhoea calf. Applying the Henderson-Hasselbalch equation to the same situation, however, suggests that it is the bicarbonate in sodium bicarbonate that is important (Constable et al., 2014). Differentiating both theories experimentally has not been achieved to date, as both theories are inextricably linked.

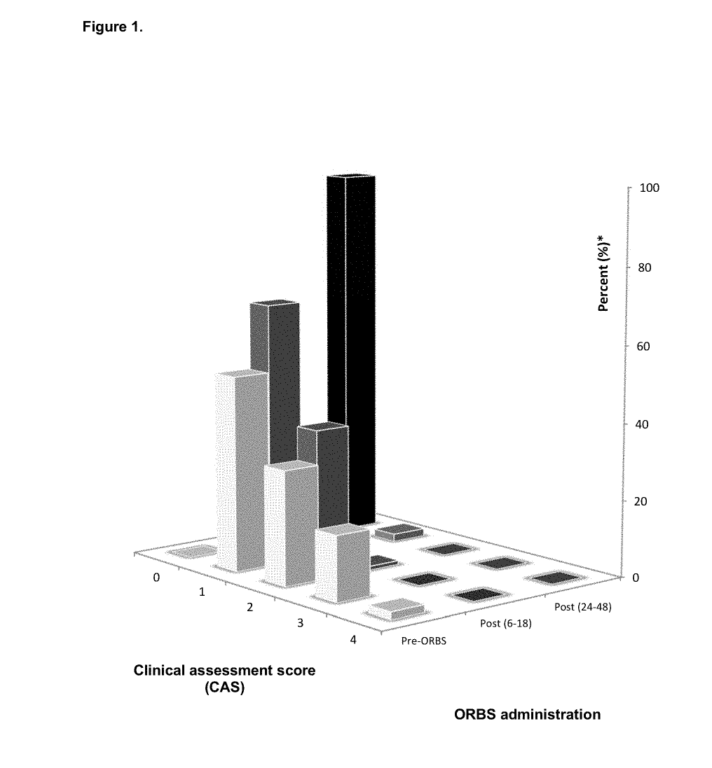

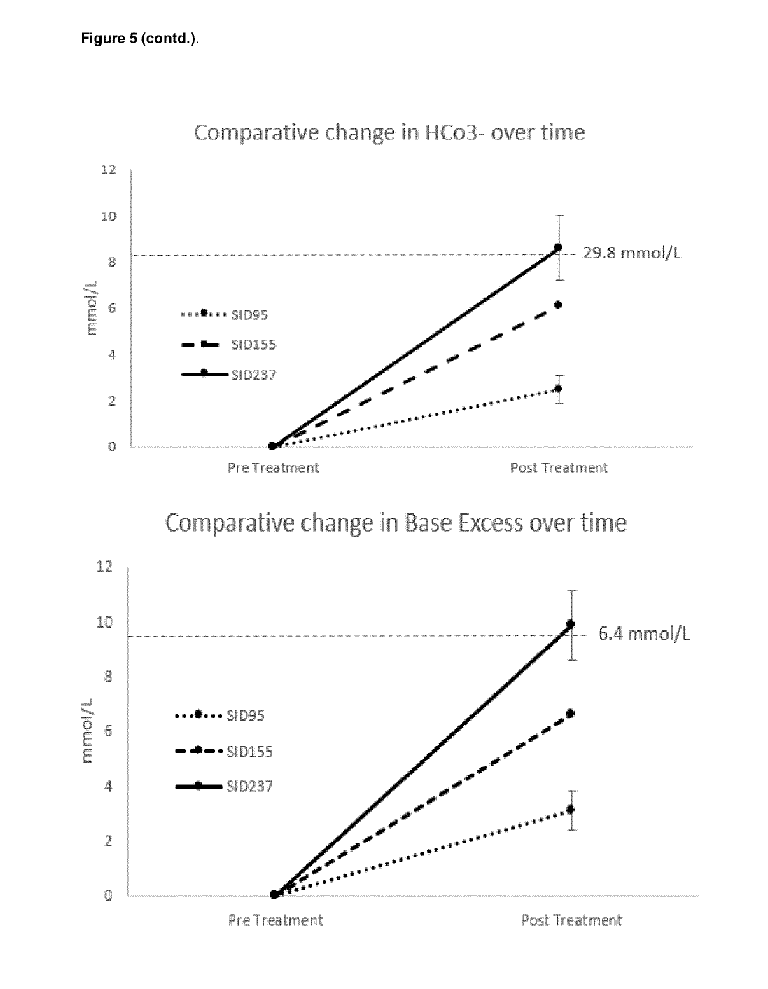

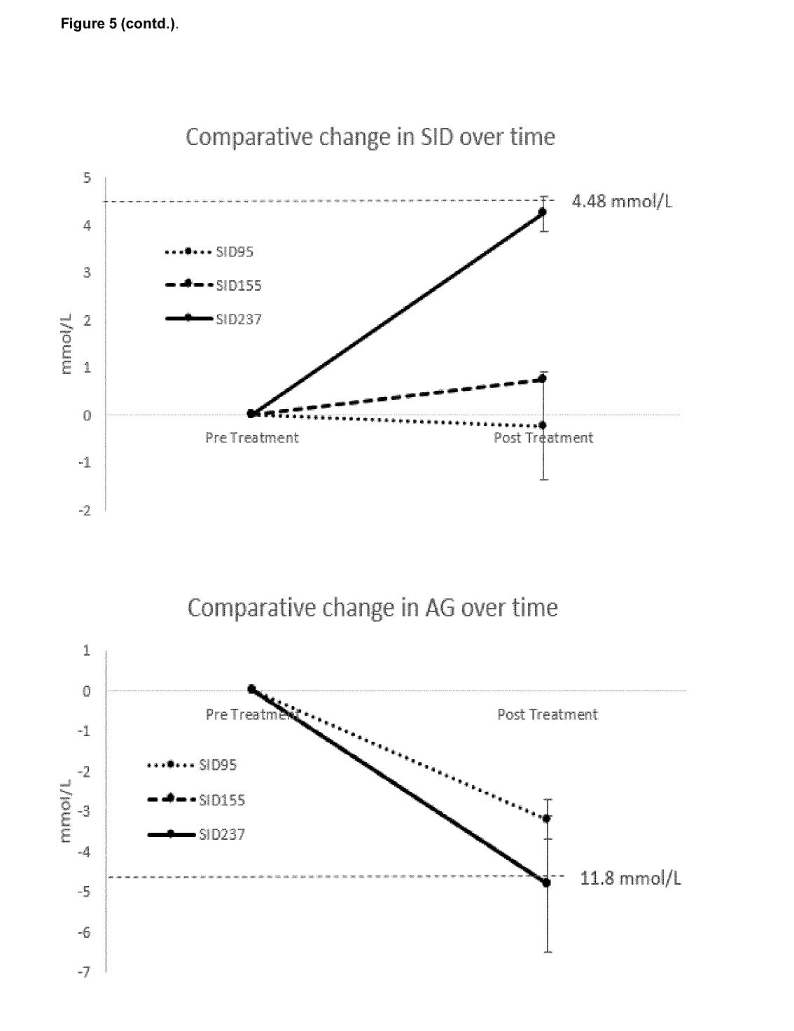

The present invention, in relation to SID and, separately, alkalising agent has greatly enhanced the efficacy of our formula relative to existing formulations. Our formula has proven to be effective with proven recovery as soon as 6 hours, representing an unprecedented improvement in recovery time, relative to the current art. FIG. 1 & FIG. 5 below demonstrate proof of this decreased time to recovery, thereby improving the animal's health and welfare and subsequently decreasing mortality.

The composition of the present invention is also effective with a two-dose treatment protocol. This is significantly less than competitor products (Appendix 2). This two-dose treatment protocol increases compliance by the end user, making it more time efficient.

The composition of the present invention has been tested against extreme cases, as determined on the basis of pH. In the study, animals with pH values as low as 6.7 were recorded (amongst the lowest values recorded in the literature), and made a full recovery with the standard treatment.

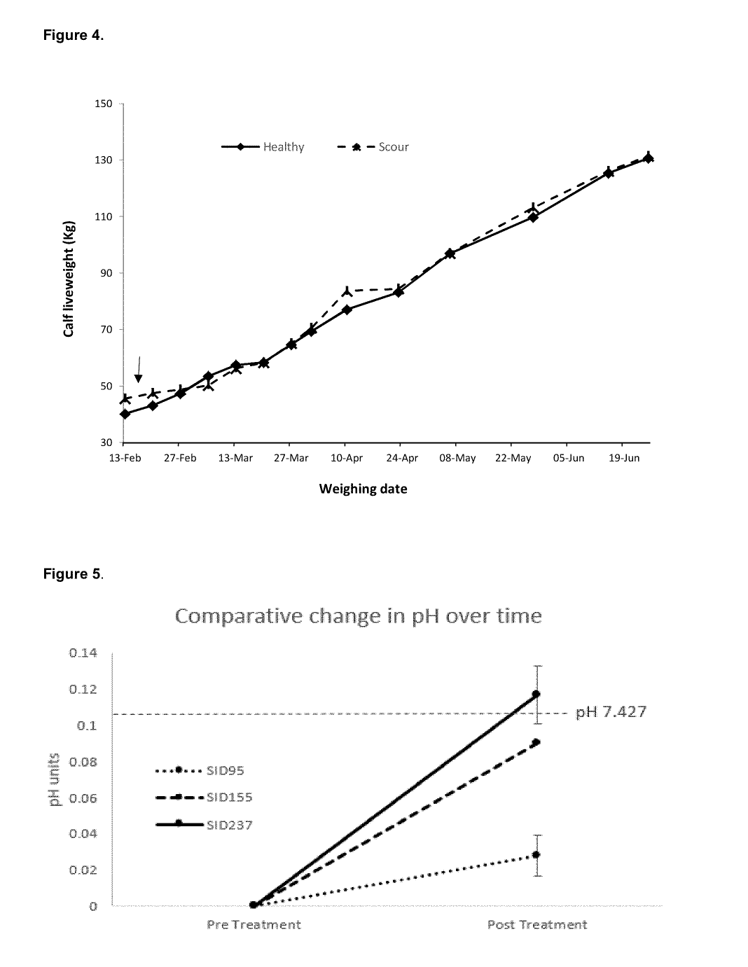

Calves treated with the composition of the present invention have a growth rate comparable to control (healthy) calves, in spite of the effects of disease (see, for example accompanying FIG. 4). The composition of the present invention enables better animal productivity and is therefore of a major economic benefit to the producer (farmer).

Optionally, the composition of the present invention may comprise tocopherol (vitamin E). Based on our knowledge and research, this vitamin has never been used for treatment of calf diarrhoea. Since vitamin E is a well-recognised antioxidant and plays an important role in calf health (Torsein et al., 2011), it is may play a role in the fast recovery using the composition of the present invention.

The composition of the present invention, while a treatment for scour in itself and exemplified hereunder in relation thereto, also has the potential for further medicinal applications where diarrhoea is also an outcome. This can be achieved by the addition of either medical (e.g. antibiotics or coccidiostats for bacterial or parasitic disease, respectively) which will further improve the efficacy this targeted market, or other ingredients (e.g. to improve palatability).

While the composition of the present invention is exemplified hereunder in relation to the treatment of calves, the composition of the present invention is also applicable to the treatment of diarrhoea in other domesticated animal species (lambs, kids, foals, piglets, dogs, cats, etc.), as well as, in humans.

By “domesticated animal” is meant animals, including animals which are or may be undergoing the process of domestication and animals that have an extensive relationship with humans beyond simple predation. This includes species which are semi-domesticated, undomesticated but captive-bred on a commercial scale, or commonly wild-caught, at least occasionally captive-bred, and tameable. Archaeozoology has identified 3 classes of animal domesticates: (1) commensals, adapted to a human niche (e.g., dogs, cats, guinea pigs); (2) prey animals sought for food (e.g., cows, sheep, pig, goats); and (3) targeted animals for draft and non-food resources (e.g., horse, camel, donkey), all of which are include in “domesticated animal”.

By “calf” is meant a bovine animal under the age of 6 months.

By “bicarbonate” is meant the anion —HCO₃⁻. By “SID”, is meant the concentration of three ions in the solution with the following formula:

[Na+]+[K+]−[Cl−]=[SID]

In the drawings,

FIG. 1. Clinical assessment scores (CAS) for diarrheic calves, pre- and post-ORBS treatment. As used herein, “ORBS” is “Vitalife”, defined hereunder.

Pre-ORBS: prior to administration of ORBS

Post (6-18): 6 to 18 hours post-administration of ORBS

Post (24-48): 24 to 48 hours post-administration of ORBS * Percentage of diarrheic case calves

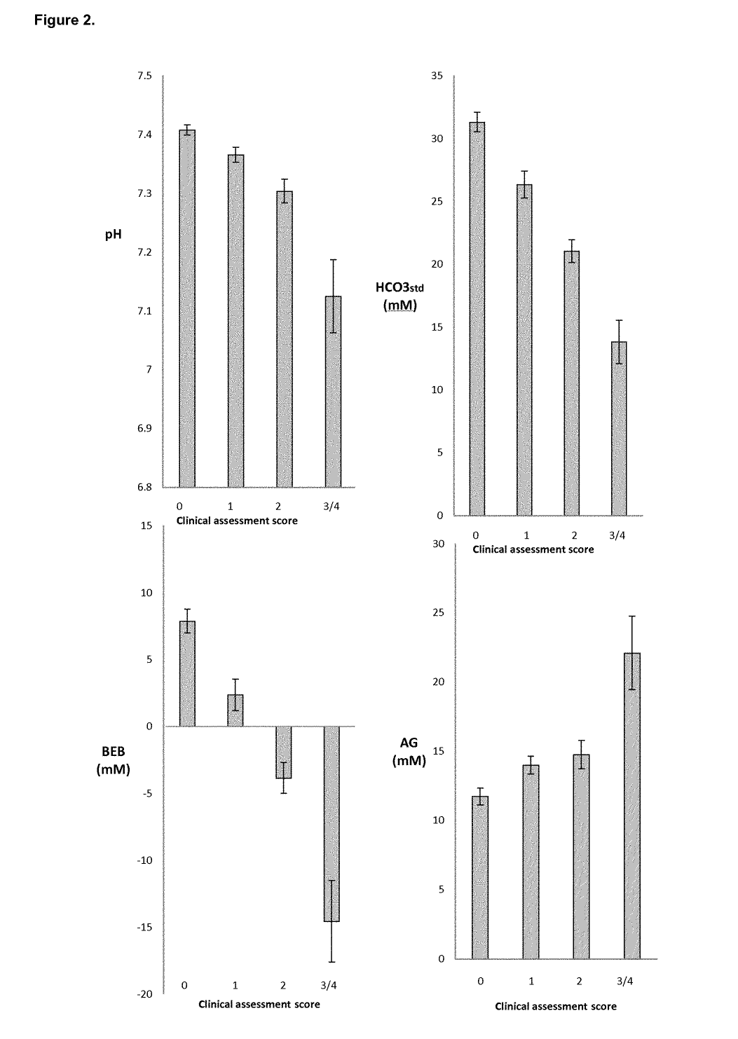

FIG. 2. Mean blood pH, HCO₃ std, base excess blood (BEB), and anion gap (AG) (±SEM) at each clinical assessment score* (CAS). *CAS groups 3 and 4 were merged for analysis. (CAS 0, n=18 (27 data points); CAS 1, n=28 (27 data points); CAS 2, n=10 (13 data points); CAS 3, n=7 (18 data points), CAS 4, n=1 (1 data point)).

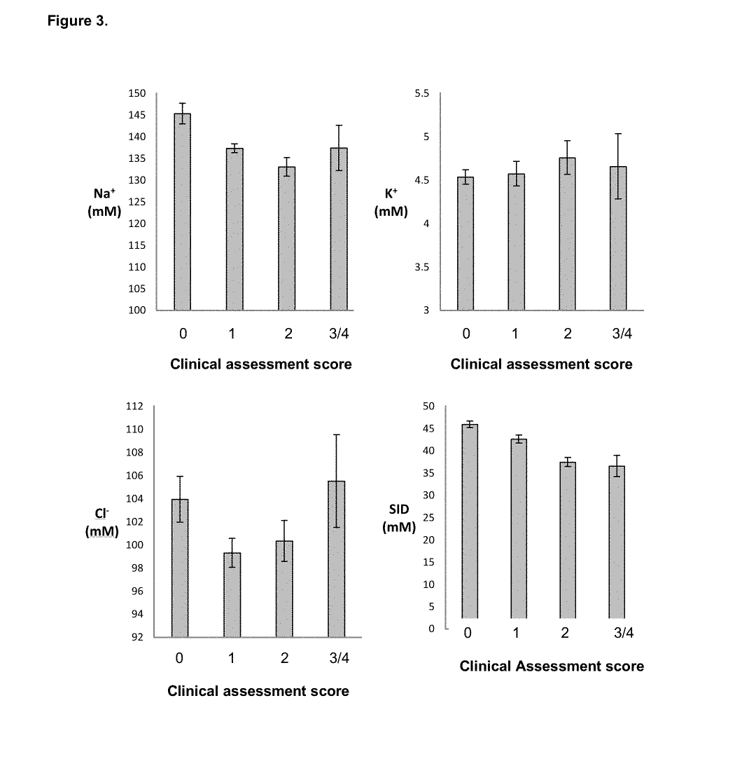

FIG. 3. Mean blood sodium (Na⁺), potassium (K⁺), Chloride (CD and strong ion difference (SID) (±SEM) at each clinical assessment score* (CAS). *CAS groups 3 and 4 were merged for analysis. (CAS 0, n=18 (27 data points); CAS 1, n=28 (27 data points); CAS 2, n=10 (13 data points); CAS 3, n=7 (18 data points), CAS 4, n=1 (1 data point)).

FIG. 4. Weight measurement over time for healthy (n=24) and ORBS-treated diarrhoea (n=8) calves.

Data records were available for farm A calf cohort only.

Arrow highlights commencement of diarrhea outbreak in this calf cohort.

FIG. 5. Comparative assessment of pH, bicarbonate (HCO₃⁻), Base Excess, SID (blood SID of the calf) and Anion Gap (AG) following administration of one of three [SID] solutions for the treatment of neonatal calf diarrhoea. Pre-treatment values were standardised by subtracting this value from the pre- and the post-treatment values. The horizontal dashed line on each graph is presented as the normal value for each variable. The three solutions had an [SID] of 95 mmol/L (n=2), [SID] of 155 mmol/L (n=1), and [SID] of 237 mmol/L (present invention) (n=2), respectively.

Appendix 1 below compares an optional embodiment of the present invention with other similar products on the market in terms of concentration of Bicarbonate, Sodium, Chloride as well as SID and osmolality.

| | | | | | Bicar- | | Osmo- |
|---|---|---|---|---|---|---|---|
| | Na⁺ | K⁺ | Cl⁻ | Dextrose | bonate | SID | lality |
| | mmol/L | mmol/L | mmol/L | mmol/L | (mmol/L) | mmol/L | mmol/L |
| Lectade Plus | 50 | 20 | 39 | 114 | 29 | 31 | 254 |
| Glutalyte | 150 | 30 | 100 | 378 | 80 | 80 | 738 |
| Present | 340 | 27 | 130 | 205 | 237 | 237 | 939 |
| Invention | | | | | | | |
| Sacrolyte | 18.5 | 7 | 33 | 161 | 24 | −7.5 | 212 |
| Life Aid Xtra | 90 | 25 | 60 | 175 | 50 | 55 | 405 |
| Effydral | 120 | 15 | 55 | 180 | 80 | 80 | 450 |
| Scourproof | 88 | 15 | 48 | | 52 | 55 | |

Hereunder, the terms “Vitalife” or “Vitalife for Calves” are used interchangeably herein to refer to a composition, when dissolved in water, that comprises:

TABLE A
“Vitalife” or “Vitalife for Calves” - an
optional embodiment of the present invention:

| | | | | Bicar- | | Osmo- |
|---|---|---|---|---|---|---|
| Na⁺ | K⁺ | Cl⁻ | Dextrose | bonate | SID | lality |
| mmol/L | mmol/L | mmol/L | mmol/L | (mmol/L) | mmol/L | mmol/L |
| 340 | 27 | 130 | 205 | 237 | 237 | 939 |

Tables below (both identified as Appendix 2) compare the dose rate and time to recovery of several different products on the market.

### Appendix 2: Number of Sachets Per Calf

| Electrolyte | Dose |
|---|---|
| “Vitalife” | 2 |
| Glutalyte | 6 |
| Lectade Plus | 6 |
| ScourProof extra | 3.5 |
| Life Aid Extra | 6 |
| Effydral | 5 (10 tablets) |
| Sacrolyte | 5 |

### Appendix 2: Days to Recovery

| Electrolyte | Time |
|---|---|
| Vitalife | <1 day |
| Glutalyte | 3 days |
| Lectade Plus | 4 days |
| ScourProof extra | 3 days |
| Life Aid Extra | 4 days |
| Effydral | 4 days |
| Sacrolyte | 3 days |

### Independent Study 1

### Materials and Methods

An observational study was conducted on dairy calves (51 healthy, 31 calves with neonatal diarrhea) during outbreaks of diarrhoea on four dairy farms. Clinical assessment scores (CAS) were assigned to each healthy and diarrhoeic calf (0 (healthy) to 4 (marked illness)). Blood gas analysis (pH, base excess (BE), Na+, K+, Ca2+, Cl−, glucose, total haemoglobin, standard HCO₃⁻, strong ion difference (SID), and anion gap (AG)) was completed for each calf. Repeated measurements were taken in healthy animals, and pre- and post-intervention measurements taken for diarrheic calves. The mean CAS of diarrheic calves was 1.7, with 51%, 30%, 17% and 2% of calves scoring 1, 2, 3 and 4, respectively. The mean values for blood pH, BE, AG and SID was 7.26, −4.93 mM, 16.3 mM and 38.59 mM, respectively. Calves were administered an oral rehydration and buffering solution (ORBS; “Vitalife”—see Table A) and reassessed. The mean CAS at 6 to 18 hours post-treatment was 0.38 (65% of calves scored 0 and 35% scored 1), which reduced to 0.03 (98% of calves scored 0 and 2% scored 1) within 24 to 48 hours. A significant increase in mean values (P<0.001) for pH, BE, HCO³−, Na+ and SID was recorded post-treatment and a significant decrease in AG, K⁺, Ca²⁺ and total haemoglobin. The correlation estimates indicated that pH, HCO₃⁻ and BE were strongly correlated with CAS, with values exceeding 0.60 in all cases (P<0.05). Administration of “Vitalife” (see Table A), an ORBS with a high SID and bicarbonate buffer, demonstrated rapid recovery from a diarrhoeic episode in dairy calves.

Clinical Assessment Score (CAS)

In order to comparatively assess diarrhoeic calves pre- and post-treatment, a five point clinical assessment scoring chart (CAS) was used. The chart was developed for use by farm managers and veterinarians at Teagasc (Irish Agriculture and Food Development Authority, Ireland) dairy research farms. Clinically healthy calves were assigned a CAS of 0, with varying degrees of ill-health scored in increments of 1, to a maximum of 4. We constructed the chart based on previously published dehydration charts (Naylor, 1989) and the Wisconsin respiratory calf health scoring model (McGurik, 2008). The chart incorporated calf demeanour, ear position, mobility, suckle reflex, enophthalmos and desire-to-feed variables. Temperature was not recorded, as the study sought to use variables most indicative of dehydration and metabolic acidosis, and variables which would also be routinely observed by producers on commercial farms. Additionally, no attempt was made to identify the underlying cause of the diarrhoea as it was not the focus of the research. Clinical assessment was completed prior to each blood sample taken, and in the case of diarrhoeic calves, an additional assessment was conducted at 24 to 48 hours post-treatment. All calves were assessed and scored simultaneously by two research veterinarians and a single consensus score recorded. All CASs were recorded prior to generation of blood gas results.

Sample Population

An observational study of 77 calves from two research (A and B) and two commercial (C and D) dairy farms was completed over a 21-day period in spring 2015. A description of husbandry regimes on each study farm for calves in the first month of life is presented in Table 1. Calves were defined as clinically healthy if they recorded a CAS of 0 (as previously described) and had no evidence of diarrhoea. Healthy calves were sampled on farm A during a period when no cases of diarrhoea had been recorded on the farm from the start of the calving season to the time of assessment (n=28, 71 measurements). Healthy case animals were also identified on farms B (n=4, 6 measurements) and C (n=19, 19 measurements) during a period of diarrhoea outbreak on those farms. Diarrhoeic case calves were defined as having a CAS of 1 or greater, and evidence of diarrhoea. Such calves were identified on farms B (n=9), C (n=2) and D (n=12). A diarrhoea outbreak subsequently occurred on farm A which facilitated analysis of an additional 8 calves, five of which were sampled earlier as part of the healthy cohort. All animals, both healthy and diarrhoeic, were enrolled to the study between the ages of 7 and 26 days.

Sampling and Administration of ORBS—“Vitalife”—see Table A

Each case calf was blood sampled by jugular venipuncture on at least one, but not more than three, occasions over the duration of the study. Venous blood samples were taken into heparinized 1 mL syringes (Cruinn Diagnostics, Dublin, Ireland), immediately placed on a bottle roller, and continuously agitated for at least 20 seconds to avoid formation of microclots. Prior to testing, all visible air bubbles were expelled from the syringe. A bench top Rapidpoint 400 (Siemens, Munich, Germany) analyzer was used to test all samples. Parameters reported by the analyzer included pH, base excess (BE; mM), Na⁺ (mM), K⁺ (mM), Ca²⁺ (mM), Cl⁻ (mM), Glucose (mM), total haemoglobin (Hb; g/dL), standard HCO₃⁻ (mM), and anion gap (AG; mM). For healthy calves, samples were taken over a period of three days, approximately two hours post-feeding. In the case of diarrhoeic calves, pre-treatment samples were taken within two hours of a milk feed being offered (many of the diarrhoeic calves had diminished suck reflexes and either fed to a limited degree or not at all). These calves were then administered an ORBS (“Vitalife”—see Table A) reconstituted in water according to manufacturer's instructions. All treatments were administered by oesophageal tube. Post-treatment blood samples were collected between six and 18 hours following ORBS intervention.

Additional Calf Data

Accurate calf date of birth, sex, breed, birth weight, whether the calf was a singleton or twin, and the level of calving difficulty experienced by the dam were available for all calves from farm A. On-going regular weight data (weekly or biweekly) were only available from farm A, and included measurement on all 8 diarrhoeic calves, and 20 of the 23 healthy calves.

Data Analysis

Data management and graphical representations were completed using Microsoft Excel (Microsoft Office 2010, Microsoft Corporation, Redmond, Wash., USA).

Preliminary steps established the stability of the variance for each of the continuous variables. For the purposes of analysis, results for calves recording CASs of 3 and 4 were grouped. Associations between various genetic and environmental factors and blood gas measurements were investigated in the healthy calf cohort from Farm A. A cross-sectional, time series, generalized estimating equation (Stata: xtgee procedure) was used to account for repeated measures. The model tested the combined effect of sex, calving difficulty, breed, date of calving, birth weight and single or twin on each of the 11 blood gas variables (pH, HCO₃⁻, BE, AG, Na⁺, K⁺, Cl⁺, Ca²⁺, Glucose, total haemoglobin and SID). Each model was constructed using a Gaussian distribution, identity link and an exchangeable correlation. The effect of ORBS treatment on the 11 blood gas measurements of diarrhoeic calves was assessed by linear regression, with status (pre- and post-ORBS treatment) used as the independent variable. The effect of environment (diarrhoea or diarrhoea-free) on the 11 blood gas measurements of healthy calves was assessed by linear regression, with environmental status used as the independent variable. A further linear regression model was constructed to assess the effect of ORBS treatment on CAS. A student t-test was used to comparatively assess calf weight at the at weekly or biweekly weight measurement time points. A Spearman correlation was used to determine the association between each blood gas variable and CAS. To achieve this, the continuous blood gas variables were reclassified as ordinal data using the standard deviation value for each variable as an increment gap size. Each increment was ranked sequentially on an ordinal scale, with 1 classed as the lowest in each case. Finally, a Pearson correlation was used to determine the association between bicarbonate and SID. P values of ≤0.05 were considered statistically significant. All statistical analysis was performed using Stata/SE v12.1 (StataCorp, Texas, USA).

Study Approval

This study was approved by the Teagasc Animal Ethics Committee (TAEC 81/2014), all procedures were authorized and carried out in accordance with the Health Products Regulatory Authority (HPRA) of Ireland (AE19132/P037).

Results

The blood gas profile of diarrhoeic calves is presented in Table 2, with healthy calf values presented for comparative purposes. The treatment of diarrhoeic calves with “Vitalife” (see Table A), an EC-compliant ORBS, led to a significant increase in mean values (P<0.001) for pH, BE, HCO₃⁻, Na⁺ and SID relative to pre-treated diarrhoeic calf values, while a significant decrease was recorded for AG, K⁺, Ca²⁺ and total Hb. None of the 31 “Vitalife”-treated animals died during the post-monitoring clinical assessment period of 8 days. On the research farms A and B where longer term records were maintained, all treated animals made a full recovery, as determined by CAS values of 0 and were returned from hospital facilities to the general calf population.

The blood gas results of healthy calves reared in a diarrhoea-free, and in a diarrhoea environment are presented in Table 3. With four exceptions (bicarbonate, SID, BE and AG), these results correspond with previously published reference ranges. Statistical comparisons between these two groups indicated that the calves reared in a diarrhoea environment had significantly lower values for pH, AG, Na⁺, Cl⁻ and glucose.

The CAS for pre- and post-ORBS (specifically “Vitalife”—see Table A) treated diarrhoeic case calves are presented in FIG. 1. The mean CAS for pre-treatment diarrhoeic calves was 1.7, with 49% of cases recording a CAS of 2 or more. Following ORBS treatment, the average CAS was reduced to 0.38, with 65% of cases recording a CAS of 0 (clinically healthy) indicating a generalized shift amongst all treated animals towards a healthy clinical status. Within 48 hours of ORBS treatment, all animals with a single exception, had a CAS value of 0 (mean CAS of 0.03).

The correlations between CAS and blood gas variables are presented in Table 4. The correlation estimates indicate that pH, HCO₃⁻, and BE were strongly and significantly correlated with CAS, with values exceeding 0.60 in all cases (P<0.05). A further correlation analysis between bicarbonate concentration and SID yielded a correlation estimate of 0.78 (P<0.0001). Graphical representations of 8 blood gas variables and CAS are presented in FIGS. 2 and 3.

Weight measurements, recorded from research farm A, in healthy and ORBS treated diarrhoeic calves, are presented in FIG. 4 and no significant difference in weights was identified at any time point (P>0.05 in all cases). Prior to the outbreak, the diarrhoea cohort had a non-significant heavier mean weight relative to the healthy cohort. In a 14-day period following the diarrhoea outbreak, this weight advantage was temporarily reversed indicating reduced growth rates in the diarrhoea cohort. However, similar mean weights were recorded thereafter for both cohorts.

The assessment of the effect of sex, calving difficulty, breed, date of calving, weight and single/twin on blood gas variables at birth indicated no significant associations.

Discussion

Blood gas analysis was used in this observational study to assess both healthy, and pre- and post-ORBS (specifically “Vitalife”—see Table A) treated diarrhoeic dairy calves. We found blood pH to be a simple and useful indicator of clinical health in study calves and would be a useful diagnostic and prognostic tool at farm level. Additionally, we observed that “Vitalife” (Table A), an ORBS which couples a high SID and a bicarbonate buffer, is an appropriate treatment for diarrhoeic calves. It effectively restored blood gas parameters to concentrations comparable to healthy animals, and all animals treated in the study recovered rapidly from the diarrhoeic episode.

The strong significant correlation we identified between CAS and pH, bicarbonate, and BE, in particular, indicates that clinical health is determined more by bicarbonate concentration than any of the electrolytes measured. This is in agreement with a number of previous studies (Geishauser and Thünker, 1997; Kasari and Naylor, 1984; Lorenz, 2004; Naylor, 1989; Wendel et al., 2001), where the link has been well established. Typically, a diarrhoeic calf will be hyponatremic, and hypo- or hyper-kalemic (Constable and Grünberg, 2013; Lewis and Phillips, 1973) based on the chronic or acute stage of the condition (Smith and Berchtold, 2014), respectively. However, the pre-treatment diarrhoeic calves in this study had a wide range of electrolyte concentrations, with no clear consensus as to a definitive hypo- or hyper-status for either sodium or potassium.

We would suggest, therefore, that for an individual diarrhoeic calf, assessment of sodium and/or potassium electrolyte concentrations is an unreliable indicator of the severity of the diarrhoea. However, the fact that bicarbonate was strongly associated with SID in these calves may support the theory that it is the intra-relationship between these elements, in addition to chloride, that is a more important associate to bicarbonate concentration rather than the individual elements themselves.

The CAS chart was used purely as a means of formalizing and standardizing assessment of calf health over the duration of this study. It is not presented, nor is it intended, to act as a validated scoring tool to inform the timing of intervention nor treatment of diarrhoeic calves. In this study, however, we have highlighted the parameters, i.e. blood pH, bicarbonate, BE, AG, that should be used in validating such a scoring system.

Assessment of healthy calves, both in a diarrhoea-free and a diarrhoeic environment, are broadly in line with previously published reference ranges (Divers and Peek, 2007; Smith, 2015). However, it should be noted that these reference values relate to adult bovine animals, as there is limited availability and variable ranges (Slanina et al., 1992) in the literature, for neonates. We identified possible exceptions to adult reference ranges published previously. For example, the lower pH reference range value of 7.31, if theoretically applied to a calf in the current study, would be considered clinically unhealthy, with a CAS of 1. Reference ranges for bicarbonate, SID and base excess were also underestimated relative to the healthy animals in this study, with AG overestimated.

The timing of blood analysis relative to feeding is a possible factor to the variations in these variables. The animals in this study were measured approximately two hours post-feeding. Age of the calf can also have a determining factor on acidaemia (Naylor, 1989), with calves during their first week of life less acidaemic than older calves. While we did not account for age in this study due to lack of accurate records on commercial study farms, the youngest diarrhoeic calf was 7 days old, thus unlikely to be naturally less acidaemic. Additionally, the fact that healthy calves reared in a diarrhoea-environment had significantly lower blood gas values, relative to those in a diarrhoea-free environment, for five of the 11 variables investigated, the possibility of management/environmental influences on blood gas parameters is raised. As further data relating to blood gas measurements for healthy neonate calves emerge from future studies, taking feeding time, neonate age (Mohri et al., 2007) and environment stressors into account, it is likely that currently reported reference ranges need to be revised. Blood gas analysis can be valuable for establishing baseline parameters, confirming a diagnosis, determining the prognosis, planning therapeutic options and monitoring response to treatment (Russell and Roussel, 2007), despite overestimating oxygen exchange fraction in some cases (Detry et al., 2003). The results of the current study support its usefulness in the field by allowing detection of electrolyte disturbance and acidosis in calves, and informative monitoring of calf recovery post-ORBS treatment. The high cost of blood gas analyzers and widespread use by veterinarians of clinical assessment alone in assessing calf diarrhoea precludes the use of this accurate diagnostic tool at farm level. The strong correlation between pH and clinical health, as measured in this study, would suggest that monitoring pH alone is useful, particularly as a measure of monitoring recovery following treatment. The availability of more simplified, economical and portable diagnostic equipment, such as a pocket blood pH meter would therefore improve accurate diagnosis and prognosis based on our results. Identifying a suitable pH cut-off value below which (further) treatment is required, would need to be established. Bleul et al. (2007) suggests a pH cut off of 7.20 to classify newborn calves as acidotic, while the lower reference range for pH is 7.31 for older animals. However, on the basis of this current analysis, a value closer to 7.36 (mean pH value for calves with a CAS value of 1) may be more appropriate for calves aged seven to 26 days. Further analysis on a larger dataset, would be of benefit in defining a suitable cut-off value.

We chose “Vitalife” (Table A) to reflect a new range of electrolyte treatments that meet with specifications of the modified EC directive. In Ireland, at least, all ORBSs must conform to this directive. The exact formulation of the ORBS used in the current study had not previously been disclosed for commercial reasons. “Vitalife” can now be disclosed as being a water-based ORBS differentiated by high SID that includes a bicarbonate buffer and additional ingredients including fat soluble vitamins. High SID alone may be sufficient as the central component of an ORBS and, when optionally combined with a buffering component, the objective of restoring calves to full health is achieved. The beneficial properties of sodium bicarbonate based buffers in ORBSs has been previously reported (Sen et al., 2009) and the results of the current study (reduction in CAS and normalisation of blood gas parameters) would suggest that coupling a high SID ORBS with a buffering component yields an effective diarrhoea treatment. However, the ORBS we used (“Vitalife”) contains additional supplements, such as tocopherol. Its contribution to the efficacy of the product cannot be disregarded in light of the important role fat soluble vitamins play in maintaining calf health (Torsein et al., 2011).

Conclusion

Administration of “Vitalife”®, an ORBS formulated on a principle of high SID, coupled to a bicarbonate buffer and supplementary nutritional ingredients, demonstrated rapid recovery from a diarrheic episode in dairy calves—the composition of Vitalife is set out in Table A. Additionally, we observed measurement of blood pH to be a useful and practical tool in monitoring calf recovery following treatment for diarrhoea.

Independent Study 2

Objective

To determine the optimal SID concentration to effectively restore deranged blood gas parameters to normal values, following a diarrhoea episode in calves.

Materials and Methods

The study was completed on a research dairy farm, where calves suffering from diarrhoea were identified. Each calf (n=7) was clinically assessed and given a clinical assessment score as per Independent Study 1. Additionally, each calf was blood sampled by jugular venepuncture for blood gas analysis. Five of the seven calves were randomly allocated into one of three groups and given one of three treatment solutions, each mixed in 2 L of warm water and administered orally. The three test solutions were:
95 mmol/L [SID] (upper range of current scientific recommendations);
155 mmol/L [SID] (the lower range of our SID claim range, and 15 mmol/L higher than the closest competitor in terms of the [SID] of a treatment solution);
237 mmol/L [SID] the [SID] of our invention.

Calves were re-evaluated 6 hours post-treatment, when another blood sample was obtained for blood gas analysis. For ease of comparison and standardisation of results, mean pre-treatment values for each treatment were subtracted from pre- and post-treatment values. This approach placed all pre-treatment values for each treatment at 0 and facilitated comparative change in the post-treatment assessment. Using two animals per group, statistical power of 0.90 was achieved, given the predicted low standard deviation in the mean difference in treatments.

Results

Of the seven calves initially assessed as having evidence of neonatal calf diarrhoea, five had a CAS of 1 and mild derangement of blood gas valuables pre-treatment. One animal was given a CAS of 0, with normal blood gas variables and another was given a CAS of 3 and had severe derangement of blood gas variables. Subsequently, only the five animals, with comparable diarrhoea severity (blood gas and clinical assessment) levels, were included in the treatment analysis.

The results are presented in FIG. 5, and indicate that the conventional scientific recommendation of an [SID] of 80-100 mmol/L is incapable of normalisation of blood gas parameters within the assessment timeframe of six hours. While the mid-range [SID] of 155 mmol/L achieved a better grade towards normalisation of the blood parameters, only Vitalife for Calves, with an [SID] of 237 mmol/L, achieves the degree of normalisation required to class a calf as healthy, both clinically and objectively with blood gas analysis within the assessment timeframe of six hours.

Discussion

The five calves with diarrhoea in this study would be classed, at the point of analysis, as ‘mild’ cases, with blood pH values not below 7.30 and moderately negative base excess values. On average, the SID₉₅ group had the lowest degree of blood gas derangement (closest to healthy levels) and SID_{Vitalife} the highest degree of derangement. That said, only Vitalife (see Table A) was able to normalise the blood gas variables and return them to pre-diarrhoea (normal) values at 6 hours post-treatment. Further assessment is required with neonatal calf diarrhoea of greater severity, however is must be noted that Vitalife for Calves (see Table A) has been documented (Independent Study 1) as correcting derangements as low as pH 6.7 within 24 hours.

Independent Study 3—Case Report

Case Report—Use of Vitalife for Calves® (See Table A) for the Treatment of a Severe Case of Neonatal Calf Diarrhoea

Objective

The objective of the visit was to manage the calf diarrhoea episode on a commercial dairy farm. This report focuses on one calf which, clinically, was regarded as the most severely affected calf with diarrhoea.

Background

A dairy farmer contacted the veterinary herd health management team regarding a severe outbreak of neonatal calf diarrhoea. The farmer reported 20 calves currently with an episode of diarrhoea, with a further 10 calves dying of neonatal calf diarrhoea within the previous 5 days. The calves in this herd were home-reared, and were managed in groups of 10 from birth, on deep straw bedding. Calves were fed milk replacer (5 L) using manual multi-calf feeding buckets.

Case Animal

A female Holstein-Friesian calf (ID: 4931), 15 days old. Reported to have commenced with diarrhoea the previous 36 hours.

Pre-Treatment Clinical Examination

The animal, on clinical examination, was depressed and unresponsive to stimulus. Ear positioning was limp. There was no evidence of a suckle reflex and no desire to feed. Eyes were severely sunken, with a corresponding estimation of dehydration of 8%. The animal stood with assistance but was unable to coordinate movement. Heart auscultation revealed bradycardia with a slight murmur on the left side. The animal was clinically moribund, and was given a CAS score of 4. A blood sample was obtained for blood gas analysis (see Table 5 for results). This blood gas picture, with particular reference to the pH, was in extremis, and amongst the lowest ever recorded in the scientific literature.

Veterinary Diagnosis

On the basis of the blood gas and clinical assessment, the animal was regarded as being severely dehydrated with a metabolic acidosis, hyponatraemia, hyperkaliaemia and hypochloridaemia.

Treatment Protocol

The calf was administered Vitalife for Calves (see Table A), administered by naso-gastric tube. No additional/ancillary treatments were administered.

Result

The animal was re-examined 18 hours later. Clinical assessment indicated a bright alert and responsive animal, with desire to feed and a good suckle reflex. Eyes were slightly sunken and ears slightly droopy. There was a willingness to walk with encouragement. The animal was given a CAS score of 1. The blood gas analysis (Table 5) indicated a recovery pattern. The animal was reassessed three hours later (T+21 h) and was given a CAS score of 1. Blood gas data for this timepoint is presented in Table 5 and indicated a near-normalisation of blood gas variables.

Conclusion

There was a marked improvement in clinical signs and evidence to support normalisation of blood gas variables within 21 hours. Long-term—follow up on this animal revealed the heifer has recently delivered a healthy calf.

TABLE 1
Description of calf husbandry regimes on each study
farm until for calves in the first month of life.

| | | | Shared | | | |
|---|---|---|---|---|---|---|
| | | | airspace | | | |
| | Predominant | | with adult | Ad lib water | Milk feeding | Creep feed |
| Farm | calf breeds | Housing | cows | available | system | available |
| A | HF | Individual calf pen | Yes | Yes | Manual multi-calf | Yes, from |
| | JeX | followed by groups | | | feeding buckets | 1 week of |
| | | pens (up to 12 | | | with an allowance | age |
| | | animals) at 3 days | | | of 6 litres milk | |
| | | of age. Deep straw | | | replacer or whole | |
| | | bedding in all pens. | | | milk per calf per | |
| | | | | | day | |
| B | HF | Individual calf pen | No | Yes | Automatic feeders | Yes, from |
| | | followed by groups | | | with an allowance | 1 week of |
| | | pens (up to 25 | | | of 6 litres milk | age |
| | | animals) at 3 days | | | replacer per calf | |
| | | of age. Deep straw | | | per day as a | |
| | | bedding in all pens. | | | routine. Isolated | |
| | | | | | and switched to | |
| | | | | | manual feeding if | |
| | | | | | diarrheic. | |
| C | HF | Deep straw bedded | No | Yes | Manual multi-calf | Yes, from |
| | JeX | group pens from | | | feeding buckets | 1 week of |
| | | birth (up to 20 | | | with an allowance | age |
| | | animals) | | | of 6 litres milk | |
| | | | | | replacer per calf | |
| | | | | | per day | |
| D | HF | Straw bedded | No | Yes | Manual multi-calf | No |
| | JeX | groups pens from | | | feeding buckets | |
| | | birth (up to 10 | | | with an allowance | |
| | | animals), moving to | | | of 4 litres milk | |
| | | woodchip bedded | | | replacer per calf | |
| | | groups pens (up to | | | per day | |
| | | 20 animals) from | | | | |
| | | approximately 2 | | | | |
| | | weeks of age. | | | | |

HF: Holstein-Friesian
JeX: Jersey cross

TABLE 2
Mean blood gas values for healthy calves and diarrhoeic calves
(pre- and post-administration of ORBS - “Vitalife” - see Table A).

| | Healthy Calves in a | | | | | |
|---|---|---|---|---|---|---|
| | diarrhoea-free environment | | Diarrhoea Calves | | | |
| | (Farm A) Value (SEM) (n = 28; | | Pre-treatment [range] (SEM) | | Post treatment [range] (SEM) | |
| Blood gas variable | 72 measurements) | | (n = 31) | | (n = 31) | |
| pH | 7.42 | (0.004) | 7.26 [6.76, 7.39] | (0.019) | 7.42 [7.28, 7.49] | (0.007)** |
| HCO₃ (mM - anion) | 29.78 | (0.311) | 20.3 [6.1, 27.1] | (0.793) | 30.9 [16.3, 38.9] | (0.763)** |
| Base Excess (mM)^{a} | 6.00 | (0.339) | −4.9 [−31.6, 3.3] | (1.173) | 7.3 [−10.3, 18.6] | (0.857)** |
| Standard | | | | | | |
| Anion Gap (mM)^{a} | 12.77 | (0.405) | 16.3 [9.2, 31.8] | (0.865) | 12.2 [5.1, 23.7] | (0.583)* |
| Na⁺ (mM) | 138.94 | (0.317) | 135.0 [118.4, 158.5] | (1.399) | 143.1 [131.4, 186.2] | (1.662)** |
| K⁺ (mM) | 4.85 | (0.040) | 4.89 [3.39, 6.59] | (0.122) | 4.32 [3.26, 5.51] | (0.018)* |
| Cl⁻ (mM) | 99.56 | (0.425) | 101.3 [84, 120] | (1.265) | 101.9 [91.0, 134.0] | (1.506) |
| SID^{b} (mM) | 44.23 | (0.411) | 38.59 [26.84, 52.18] | (0.819) | 45.53[35.43, 56.92] | (0.658)** |
| Glucose (mM) | 7.91 | (0.359) | 5.2 [2.5, 8.3] | (0.178) | 5.5 [3.7, 8.8] | (0.180) |
| Ca²⁺ (mM) | 1.26 | (0.007) | 1.27 [1.14, 1.42] | (0.012) | 1.20 [1.01, 1.33]** | (0.012) |
| Total Hb (g/dL) | 11.67 | (0.183) | 13.5 [7.7, 20.0] | (0.438) | 12.1 [9.6, 15.7] | (0.276)* |

Statistical difference between pre- and post-treatment values estimated using linear regression,
*P = 0.001,
**P < 0.0001
^{a}Calculated using blood gas machine algorithm;
^{b}SID = [Na+] + [K+] − [Cl−]

TABLE 3
Mean blood gas values for healthy calves in a diarrhoea and diarrhoea-free environment.

| | Healthy Calf | | | | |
|---|---|---|---|---|---|
| | | | Diarrhea environment | | |
| | Diarrhea-free environment | | (Farms B, C &D) Value | | |
| | (Farm A) Value (SEM) | | (SEM) (n = 23; 25 | | Reference |
| Blood gas variable | (n = 28; 71 measurements) | | measurements) | | ranges^{cde} |
| pH | 7.42 | (0.004) | 7.39 | (0.006)* | 7.31-7.53^{c} |
| HCO₃ (mM - anion) | 29.78 | (0.311) | 30.19 | (0.465) | 17-29^{c} |
| Base Excess (mM)^{a} | 6.00 | (0.339) | 7.09 | (0.502) | 0 ± 2^{e} |
| Standard | | | | | |
| Anion Gap (mM)^{a} | 12.77 | (0.405) | 10.27 | (0.429)* | 14-20^{c} |
| Na⁺ (mM) | 138.94 | (0.317) | 135.88 | (0.617)* | 132-152^{c} |
| K⁺ (mM) | 4.85 | (0.040) | 4.73 | (0.096) | 3.9-5.8^{c} |
| Cl⁻ (mM) | 99.56 | (0.425) | 96.64 | (0.553)* | 97-111^{c} |
| SID^{b} (mM) | 44.23 | (0.411) | 43.96 | (0.457) | 38-42^{c} |
| Glucose (mM) | 7.91 | (0.359) | 5.71 | (0.185)* | 2.49-4.16^{c} |
| Ca²⁺ (mM) | 1.26 | (0.007) | 1.24 | (0.011) | >1.0^{d} |

| | | | | |
|---|---|---|---|---|
| Total Hb (g/dL) | 11.67 | (0.183) | — | 8.6-11.9^{d} |

Statistical difference between pre- and post-treatment values estimated using linear regression,
*P = 0.001
^{a}Calculated using blood gas machine algorithm
^{b}SID = [Na+] + [K+] − [Cl−]
^{c}Adult ranges from Smith (2015)
^{d}Adult ranges from Divers and Peek (2007)
^{e}Adult ranges from Stampfli et al. (2012).

TABLE 4
Spearman correlation coefficients between blood gas variables
(reclassified as ordinal data) and calf clinical assessment score
for all diarrhoeic study calves.

| Blood gas variable | Spearman correlation coefficient (rho) |
|---|---|
| pH | −0.63* |
| HCO_{3 (}as anion) | −0.75* |
| Base Excess Standard | −0.74* |
| Anion Gap | 0.40* |
| Na⁺ | −0.39* |
| K⁺ | 0.11 |
| Cl⁻ | −0.03 |
| SID^{a} | −0.59* |
| Glucose | −0.30* |
| Ca²⁺ | −0.12 |
| Total Hb | 0.23* |

*P < 0.05
^{a}SID = [Na⁺] + [K⁺] − [Cl⁻]

TABLE 5
Mean blood gas values for healthy calves and one diarrhoeic case
calf (pre- and post-administration of Vitalife for Calves - see Table A).

| | | Case Calf (4931) | | |
|---|---|---|---|---|
| | | Pre- | Post | |
| | Normal | treatment | treatment | |
| | values (SEM) | (T0, | (T + 18 h, | Post |
| Blood gas | (n = 28; 72 | followed by | followed | treatment |
| variable | measurements)^{c} | sachet 1) | by sachet 2) | (T + 21 h) |
| pH | 7.42 (0.004) | 6.76 | 7.28 | 7.39 |
| HCO₃ | 29.78 (0.311) | 6.1 | 16.3 | 31.8 |
| (mM as | | | | |
| anion) | | | | |
| Base | 6.00 (0.339) | −31.6 | −10.3 | 8.5 |
| Excess | | | | |
| (mM)^{a} | | | | |
| Anion | 12.77 (0.405) | 22.8 | 23.7 | 8.7 |
| Gap | | | | |
| (mM)^{a} | | | | |
| Na⁺ (mM) | 138.94 (0.317) | 135.3 | 156 | 131.4 |
| K⁺ (mM) | 4.85 (0.040) | 6.54 | 4.01 | 3.26 |
| Cl⁻ (mM) | 99.56 (0.425) | 115 | 121 | 91 |
| SID^{b} (mM) | 44.23 (0.411) | 26.84 | 39.01 | 43.66 |
| Glucose | 7.91 (0.359) | 5.7 | 6.9 | 4.0 |
| (mM) | | | | |
| Ca²⁺ (mM) | 1.26 (0.007) | 1.31 | 1.02 | 1.12 |
| Total Hb | 11.67 (0.183) | 19.4 | 15.4 | 10.9 |
| (g/dL) | | | | |

^{a}Calculated using blood gas machine algorithm;
^{b}SID = [Na+] + [K+] − [Cl−]
^{c}Adopted from Independent study 1.

### REFERENCES

Azizzadeh, M., Shooroki, H. F., Kamalabadi, A. S., Stevenson, M. A. 2012. Factors affecting calf mortality in Iranian Holstein dairy herds. Prey. Vet. Med. 104, 335-340.
Bleul, U., Lejeune, B., Schwantag, S., Kahn, W. 2007. Blood gas and acid-base analysis of arterial blood in 57 newborn calves. Vet. Rec. 161, 688-691.
Bachmann, L., Homeier, T., Arlt, S., Brueckner, M., Rawel, H., Deiner, C., Hartmann, H. 2009. Influence of different oral rehydration solutions on abomasal conditions and the acid-base status of suckling calves. J. Dairy. Sci. 92, 1649-1659.
Bachmann, L., Schmidt, B., Ruwolf, U., Wenge, J., Coenen, M. 2012. Change of plasma volume, osmolality, and acid-base status in healthy calves after feeding of milk and water- and milk-based oral rehydration solutions, J. Dairy. Sci. 95, 6006-6014.
Constable P D. 2000. Clinical assessment of acid-base status: comparison of the Henderson-Hasselbalch and strong ion approaches. Vet Clin Pathol 29, 115-28.
Constable, P. D., Grunberg, W., Carstensen, L. 2009. Comparative effects of two oral rehydration solutions on milk clotting, abomasal luminal pH, and abomasal emptying rate in suckling calves. J. Dairy. Sci. 92, 296-312.
Constable, P. D., Grünberg, W. 2013. Hyperkalemia in diarrheic calves: Implications for diagnosis and treatment. Vet. J. 3, 271-272.
Detry, B., Cambier, C., Frans, A., Gustin, P., Clerbaux, T. 2003. Calculation of bovine haemoglobin oxygen saturation by algorithms integrating age, haemoglobin content, blood pH, partial pressures of oxygen and carbon dioxide in the blood, and temperature. Vet. J. 165, 258-265.
Divers, T. J., Peek, S. 2007. The Clinical Examination. In: Rebhun's diseases of dairy cattle, Second Ed. Elsevier, St. Louis, Mo., USA, pp. 14.
Geishauser, T., Thünker, B. 1997. Metabolic acidosis in diarrheic neonatal calves-estimation using suckling reflex and standing ability. Prakt. Tierarzt. 78, 600-605.
Grunberg, W., Hartmann, H., Arlt, S., Burfeind, O., Staufenbiel, R. 2013. Alkalinizing effect of NaHCO(3) with and without glucose when administered orally to euhydrated neonatal dairy calves. J. Dairy. Sci. 96, 3895-3906.
Kasari, T., Naylor, J. 1984. Metabolic acidosis without clinical signs of dehydration in young calves. Can. Vet. J. 25, 394.
Kirchner, D., Schwedhelm, L., Coenen, M., Bachmann, L. 2014. Dietary influences on the hydration and acid-base status of experimentally dehydrated dairy calves. Vet. J. 199, 251-257.
Lewis, L., Phillips, R. 1973. Diarrheic induced changes in intracellular and extracellular ion concentrations in neonatal calves. In: Annales de Recherches Vétérinaires, pp. 99-111.
Lorenz, I., Fagan, J., More, S. J. 2011. Calf health from birth to weaning. II. Management of diarrhea in pre-weaned calves. Ir. Vet. J. 64, 10.1186.
Lorenz, I. 2004. Investigations on the influence of serum D-lactate levels on clinical signs in calves with metabolic acidosis. Vet. J. 168, 323-327.
McGuirk, S. M. 2008. Disease Management of Dairy Calves and Heifers. Vet. Clin. North Am. Food Anim. Pract. 24, 139-153
Meganck, V., Hoflack, G., Opsomer, G. 2014. Advances in prevention and therapy of neonatal dairy calf diarrhea: a systematical review with emphasis on colostrum management and fluid therapy. Acta Vet. Scand. 56, 75.
Mohri, M., Sharifi, K., Eidi, S. 2007. Hematology and serum biochemistry of Holstein dairy calves: age related changes and comparison with blood composition in adults. Res. Vet. Sci. 83, 30-39.
Naylor, J. M. 1989. A retrospective study of the relationship between clinical signs and severity of acidosis in diarrheic calves. Can. Vet. J. 30, 577.
Russell, K. E., Roussel, A. J. 2007. Evaluation of the ruminant serum chemistry profile. Vet. Clin. North Am. Food Anim. Pract. 23, 403-426.
Sen, I., Altunok, V., Ok, M., Coskun, A., Constable, P. D. 2009. Efficacy of oral rehydration therapy solutions containing sodium bicarbonate or sodium acetate for treatment of calves with naturally acquired diarrhea, moderate dehydration, and strong ion acidosis. J. Am. Vet. Med. Assoc. 234, 926-934.
Slanina, L., Nagy, O., Sedovic, M., Struharikova, J. 1992. Dynamics of the acid-base balance of venous and arterial blood in clinically healthy calves. DTW. Dtsch. Tierarztl. Wochenschr. 99, 182-186.
Smith, B. P. 2015. Clinical Chemistry Tests. In: Large Animal Internal Medicine, Fifth ed. Elsevier, St. Louis, Mo., USA.
Smith, G. W., 2009. Treatment of Calf Diarrhea: Oral Fluid Therapy. Vet. Clin. North Am. Food Anim. Pract. 25, 55-72.
Smith, G. W., Berchtold, J. 2014. Fluid Therapy in Calves. Vet. Clin. North Am. Food Anim. Pract. 30, 409-427.
Stämpfli, H., Oliver, O., Pringle, J. K. 2012. Clinical evaluation of an oral electrolyte solution formulated based on strong ion difference (SID) and using propionate as the organic anion in the treatment of neonatal diarrheic calves with strong ion acidosis. Open. J. Vet. Med. 2, 34-39.
Stämpfli, H., Pringle, J., Lumsden, J., Oliver, O., Kilmer, B., Baird, J. 1996. Experimental evaluation of a novel oral electrolyte solution in the treatment of natural occurring neonatal calf diarrhea. Proceedings of the XIX World Buiatrics Congress, Edinburgh, UK.
Stewart, P. A. 1981. Strong ions, plus carbon dioxide, plus weak acid, isolated blood plasma and isolated intracellular fluid. In: How to understand acid-base, Elsevier, New York.
Torsein, M., Lindberg, A., Sandgren, C. H., Waller, K. P., Törnquist, M., Svensson, C. 2011. Risk factors for calf mortality in large Swedish dairy herds. Prey. Vet. Med. 99, 136-147.
Wendel, H., Sobotka, R., Rademacher, G. 2001. Study on the correlation between blood acidosis and clinical signs in calves with neonatal diarrhea. Tierarztl. Umsch. 56, 351-356.

The invention is not limited to the embodiments described herein but can be amended or modified without departing from the scope of the present invention.

1. A pharmaceutical or veterinary composition comprising a strong ion difference in the range of about 150-370 mmol/l, optionally about 165-370 mmol/l, when dissolved in a diluent, optionally water, further optionally water having a strong ion difference of less than about 10 mmol/l.

2. The composition of claim 1 comprising a strong ion difference in the range of about 150-270 mmol/l, when dissolved in a diluent, optionally water, further optionally water having a strong ion difference of less than about 10 mmol/l.

3. The composition of claim 1 comprising a strong ion difference in the range of about 270-370 mmol/l, when dissolved in a diluent, optionally water, further optionally water having a strong ion difference of less than about 10 mmol/l.

4. The composition of claim 1 comprising a strong ion difference in the range of about 200-370 mmol/l, when dissolved in a diluent, optionally water, further optionally water having a strong ion difference of less than about 10 mmol/l.

5. The composition of claim 2 comprising a strong ion difference in the range of about 200-270 mmol/l, when dissolved in a diluent, optionally water, further optionally water having a strong ion difference of less than about 10 mmol/l.

6. A pharmaceutical or veterinary composition comprising, when dissolved in a diluent, optionally water, an alkalising agent, optionally bicarbonate, in the range of about 130 to 370 mmol/l, optionally about 130 to 300 mmol/l, when dissolved in a diluent, optionally water, further optionally water having a strong ion difference of less than about 10 mmol/l.

7. The composition of claim 6 comprising an alkalising agent, optionally bicarbonate, in the range of about 150 to 370 mmol/l, optionally about 130 to 300 mmol/l, when dissolved in a diluent, optionally water, further optionally water having a strong ion difference of less than about 10 mmol/l.

8. The composition of claim 6 or 7, comprising, when dissolved in the diluent, a strong ion difference in the range of about 150-270 mmol/l.

9. The composition of any one of claims 1 to 5, comprising, when dissolved in the diluent, an alkalising agent, optionally bicarbonate, in the range of about 130 to 370 mmol/l, optionally about 130 to 300 mmol/l.

10. The composition of any one of claims 1 to 5, comprising, when dissolved in the diluent, an alkalising agent, optionally bicarbonate, in the range of about 150 to 370 mmol/l, optionally about 150 to 300 mmol/l.

11. The composition of any one of claims 1 to 5, 9 and 10 comprising, when dissolved in the diluent, an alkalising agent, optionally bicarbonate, in the range of about 300 to 370 mmol/l.

12. The composition of any one of claims 1 to 5, 9 and 10 comprising, when dissolved in the diluent, an alkalising agent, optionally bicarbonate, in the range of about 150 to 370 mmol/l.

13. The composition of claim 6 or 7, comprising, when dissolved in the diluent, a strong ion difference in the range of about 150-370 mmol/l, optionally a strong ion difference in the range of about 200-350 mmol/l, further optionally a strong ion difference in the range of about 165-370 mmol/l, optionally a strong ion difference in the range of about 270-370 mmol/l, optionally a strong ion difference in the range of about 150-270 mmol/l.

14. The composition of any one of claims 1 to 13, in which the sodium ion concentration, when dissolved in the diluent, is in the range about 190 to 390 mmol/l, optionally about 250 to 375 mmol/l, further optionally about 340 mmol/l.

15. The composition of any one of claims 1 to 13, in which the sodium ion concentration, when dissolved in the diluent, is in the range about 250 to 480 mmol/l, optionally about 190 to 480 mmol/l, further optionally about 250 to 430 mmol/l, still further optionally about 340 mmol/l.

16. The composition of any one of claims 1 to 15, in which the potassium ion concentration, when dissolved in the diluent, is in the range about 20 to 40, optionally about 27 mmol/l.

17. The composition of any one of claims 1 to 16, in which the chloride ion concentration, when dissolved in the diluent, is in the range about 60 to 150 mmol/l, optionally about 75 to 145 mmol/l, further optionally about 130 mmol/l.

18. The composition of any one of claims 1 to 16, in which the chloride ion concentration, when dissolved in the diluent, is in the range about 60 to 150 mmol/l, optionally about 75 to 145 mmol/l, further optionally about 115 to 145 mmol/l, still further optionally about 130 mmol/l.

19. The composition of any one of claims 6 to 8 and 13, in which the alkalising agent, when dissolved in the diluent, is in the range of about 175 to 300 mmol/l, optionally about 200 to 300 mmol/l, further optionally about 237 mmol/l.

20. The composition of any one of claims 6 to 8, 13 and 19, in which the alkalising agent, when dissolved in the diluent, is in the range of about 150 to 370 mmol/l, optionally about 200 to 370 mmol/l, further optionally about 237 mmol/l.

21. The composition of any one of claims 6 to 20, in which the alkalising agent, when dissolved in the diluent, is selected from bicarbonate (HCO₃) or a bicarbonate precursor optionally selected from propionate, acetate or citrate; in which, optionally, in which the alkalising agent is bicarbonate (HCO₃).

22. The composition of claim 21, in which the alkalising agent is bicarbonate (HCO₃) that, when dissolved in the diluent, is in the range of about 175 to 300 mmol/l, optionally about 200 to 300 mmol/l, further optionally about 237 mmol/l.

23. The composition of claim 21, in which the alkalising agent is bicarbonate (HCO₃) that, when dissolved in the diluent, is in the range of about 150 to 370 mmol/l, optionally about 200 to 370 mmol/l, further optionally about 237 mmol/l.

24. The composition of any one of claims 1 to 23, the composition comprising an osmolality in the range of about 750 to 1000 mOsm/l, optionally about 900 to 1000 mOsm/l, further optionally about 939 mOsm/l.

25. The composition of any one of claims 1 to 23, the composition comprising an osmolality in the range of about 750 to 1300 mOsm/l, optionally about 900 to 1300 mOsm/l, further optionally about 939 mOsm/l.

26. The composition of any one of claims 1 to 5, 9 to 12, 14 to 18 and 21 to 25, in which the strong ion difference, when dissolved in the diluent, is in the range of about 170 to 270 mmol/l, optionally about 215 to 255 mmol/l, further optionally about 237 mmol/l.

27. The composition of any one of claims 1 to 5, 9 to 12, 14 to 18 and 21 to 2612, in which the strong ion difference, when dissolved in the diluent, is in the range of about 150 to 370 mmol/l, optionally about 200 to 370 mmol/l, further optionally about 215 to 300 mmol/l, still further optionally about 237 mmol/l.

28. The composition of any one of claims 1 to 27, further comprising a water soluble vitamin, optionally, tocopherol.

29. A method of treating diarrhoea in a subject, optionally a domesticated animal, the method comprising administering the composition of any one of claims 1 to 28.

30. The method of claim 29, in which the subject is a domesticated animal that is optionally selected from calves, lambs, kids, foals, piglets, dogs, cats, further optionally is calves.

31. The method of claim 29 or 30, in which the composition is administered in a one dose treatment protocol or, alternatively, in a two dose treatment protocol administered about 12 hours apart.

32. The method of claim 31, in which the, or each, dose is provided in a volume in the range of 0.25 to 5 litres.

33. The method of claim 31, in which the subject is a calf and the, or each, dose is provided in a volume in the range of about 1 to 3.5 litres, optionally about 2 litres.

34. The method of any one of claims 29 to 33, in which the composition is administered without either milk or milk replacer.

35. The method of any one of claims 29 to 34, in which the composition is administered per os.

36. The pharmaceutical or veterinary composition of any one of claims 1 to 28 dissolved in about 0.25 to 5 litres of diluent.

37. The pharmaceutical or veterinary composition of any one of claims 1 to 28 dissolved in about 1 to 3.5 litres, optionally about 2 litres of diluent.

38. The pharmaceutical or veterinary composition of any one of claim 1 to 28, 37 or 38, comprising a strong ion difference of about 237 mmol/l, when dissolved in water, optionally water having a strong ion difference of less than about 10 mmol/l.

39. The pharmaceutical or veterinary composition of claim 38, comprising, when dissolved in water, optionally water having a strong ion difference of less than about 10 mmol/l:

| Na⁺ | | | SID |
|---|---|---|---|
| mmol/L | K⁺ mmol/L | Cl⁻ mmol/L | mmol/L |
| 340 | 27 | 130 | 237 |

40. The pharmaceutical or veterinary composition of claim 38 or 39, further comprising about 237 mmol/L of Bicarbonate.

41. The pharmaceutical or veterinary composition of any one of claims 38 to 40, further comprising about 205 mmol/L of Dextrose.

42. The pharmaceutical or veterinary composition of any one of claims 38 to 41, having an osmolality of about 939 mmol/L.

43. The pharmaceutical or veterinary composition of any one of claims 1 to 28 or 37 to 42, consisting of, when dissolved in water, optionally water having a strong ion difference of less than about 10 mmol/l:

| | | | | Bicar- | | Osmo- |
|---|---|---|---|---|---|---|
| Na⁺ | K⁺ | Cl⁻ | Dextrose | bonate | SID | lality |
| mmol/L | mmol/L | mmol/L | mmol/L | (mmol/L) | mmol/L | mmol/L |
| 340 | 27 | 130 | 205 | 237 | 237 | 939 |

44. A composition of any one of claims 1 to 28 or 36 to 43 for use in a method of treating diarrhoea in a subject, optionally a domesticated animal, the use comprising administering the composition of any one of claims 1 to 28 or 36 to 43.

45. The composition for use of claim 44 in which the subject is a domesticated animal that is optionally selected from calves, lambs, kids, foals, piglets, dogs, cats, further optionally is calves.

46. The composition for use of claim 44 or 45, in which the composition is administered in a one dose treatment protocol or, alternatively, in a two dose treatment protocol administered about 12 hours apart.

47. The composition for use of claim 44 or 45, in which the, or each, dose is provided in a volume in the range of about 0.25 to 5 litres.

48. The composition for use of claim 45, in which the subject is a calf and the, or each, dose is provided in a volume in the range of about 1 to 3.5 litres, optionally about 2 litres.

49. The composition for use of any one of claims 44 to 48, in which the composition is administered without either milk or milk replacer.

50. The composition for use of any one of claims 44 to 49, in which the composition is administered per os.
